(19) United States
(12) Patent Application Publication
(10) Pub. No.: US 2015/0112180 A1
(43) Pub. Date: Apr. 23, 2015

(54) MESOPOROUS NEURONAL ELECTRODE USING SURFACTANT AND METHOD OF MAKING THE SAME

(71) Applicant: Electronics and Telecommunications Research Institute, Daejeon (KR)

(72) Inventors: Yong Hee KIM, Daejeon (KR); Nam Seob BAEK, Daejeon (KR); Sang Don JUNG, Daejeon (KR)

(73) Assignee: Electronics and Telecommunications Research Institute, Daejeon (KR)

(21) Appl. No.: 14/288,213

(22) Filed: May 27, 2014

(30) Foreign Application Priority Data
Oct. 23, 2013 (KR) 10-2013-0126402

(51) Int. Cl. A61B 5/04; C25D 13/02; C25D 13/12

(52) U.S. Cl. CPC A61B 5/04001; C25D 13/12; C25D 13/02
USPC 600/377; 204/490

(57) ABSTRACT

A mesoporous neuronal electrode using a surfactant and a method of making the same are disclosed. A mesoporous neuronal electrode according to an exemplary embodiment includes a first metal nanoparticle, a second metal nanoparticle or both of the first and second metal nanoparticles on a surface of the electrode.

### CROSS-REFERENCE TO RELATED APPLICATION

This application claims the priority benefit of Korean Patent Application No. 10-2013-0126402, filed on Oct. 23, 2013, in the Korean Intellectual Property Office, the disclosure of which is incorporated herein by reference.

### BACKGROUND

1. Field of the Invention The present invention relates to a mesoporous neuronal electrode using a surfactant and a method of making the same.

2. Description of the Related Art

A neuronal electrode is used to record a neural signal with a broad amplitude by approaching a neuron or a neural stem and to stimulate neuronal cell in vivo or in vitro neural interfaces. Measurements of neuronal signals have been developing mostly with neurophysiological studies in diverse areas of from membrane potentials of neuronal cells to peripheral nerves, spinal nerves and cranial nerves. To record tiny neural signals with a high signal-to-noise ratio, the neural electrode should have low impedance since the noise level is proportional to the impedance.

As materials used for the neuronal electrode, a first-generation electrode made of metal wires such as white gold, gold, tungsten and iridium and a second-generation electrode such as a semiconductor and a multi-electrode array are widely employed.

To accurately identify the state of a neuronal cell, it is necessary to record neural signals by each nerve cell. The size of the electrode used for long-term extracellular recording is gradually decreasing; it is for avoiding interference from neighboring electrode. However, it is well-known that the electrode impedance is inverse proportion to electrode surface area. Thus in order to increase surface area of electrode, surface modification with nanomaterial is needed.

### SUMMARY

An aspect of the present invention is to provide a mesoporous neuronal electrode having a large surface area.

Another aspect of the present invention is also to provide a method of making a mesoporous neuronal electrode that is surface-modified with different kinds of mesoporous nanoparticles, which are combined with a bio-affinitive functional group.

Still another aspect of the present invention is to provide an apparatus for measuring a mesoporous neuronal electrode which includes a mesoporous neuronal electrode having a low impedance and a high capacitance.

According to an aspect of the present invention, there is provided a mesoporous neuronal electrode including a nanoparticle layer being formed on a surface of the electrode and including at least one of a first metal nanoparticle and a second metal nanoparticle selected from the group consisting of a nanotube, a hollow nanoparticle and a nanowire.

The nanoparticle layer may have a thickness of 40 nm to 1 μm.

A first metal may include gold and a second metal may include white gold, or the first metal may include white gold and the second metal may include iridium.

The mesoporous neuronal electrode may have an impedance of 1×10⁷ or lower at 1 kHz.

The mesoporous neuronal electrode may have a capacitance of 1 mF/Cm² or higher.

The first metal nanoparticle, the second metal nanoparticle or both of the first metal nanoparticle and the second metal nanoparticle may be combined with a functional group.

The functional group may be combined via self-assembly with the first metal nanoparticle, the second metal nanoparticle or both of the first metal nanoparticle and the second metal nanoparticle.

The functional group may be a bioaffinitive functional group.

The functional group may include a thiol group.

According to another aspect of the present invention, there is provided a neural signal measuring apparatus including the mesoporous neuronal electrode.

According to still another aspect of the present invention, there is provided a method of making a mesoporous neuronal electrode, the method including preparing a mixture solution including a first metal precursor solution, a second metal precursor solution and a surfactant; introducing a target electrode into the mixture solution and electro-co-depositing a first metal nanoparticle and a second metal nanoparticle on a surface of the target electrode; and washing the target electrode with the electro-co-deposited first metal nanoparticle and second metal nanoparticle to remove the surfactant and to form a carbon nanotube, a hollow nanoparticle or both of the carbon nanotube and the hollow nanoparticle including the first metal nanoparticle and the second nanoparticle.

A first metal precursor may include HAuCl₄ or KAuCl₄, and a second metal precursor may include H₂PtCl₆ or K₂PtCl₆.

The surfactant may be present in an amount of 0.1 to 30% by weight (wt %) in the mixture solution.

The surfactant may include at least one selected from the group consisting of P123, SDS, CTAB and Triton X.

The electro-co-depositing may be carried out by cyclic voltammetry or a constant voltage method.

The method may further include introducing a function group to be combined with the first metal nanoparticle, the second metal nanopaticle or both of the first metal nanoparticle and the second metal nanoparticle after the washing.

### EFFECTS OF THE INVENTION

According to embodiments of the present invention, there is provided a mesoporous neuronal electrode having a low impedance and a high capacitance.

According to embodiments of the present invention, there is provided a method of making a mesoporous neuronal electrode that is surface-modified with different kinds of mesoporous nanoparticles and thus has a large specific surface area.

According to embodiments of the present invention, it is possible to effectively make a mesoporous neuronal electrode that is surface-modified with different kinds of nanoparticles and by combining a functional group with the nanoparticles.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a scanning electron microscopy (SEM) of gold/white gold nanoparticles according to an example of the present invention;

FIG. 2(a) is a light microscope image of a mesoporous neuronal electrode array with electro-co-deposited gold/white gold nanoparticles according to the example of the present invention;

FIG. 2(b) is a SEM image of one electrode of electrode array according to the FIG. 2(a);

FIG. 2(c) is a SEM image of nanoparticles deposited in electrode according to the FIG. 2(b);

FIG. 3 is a graph illustrating impedances of neuronal electrodes according to the example and comparative examples 1 to 3;

FIG. 4 is a graph illustrating capacitances of the neuronal electrodes according to the example and the comparative examples 1 to 3; and

FIG. 5 schematically illustrates the mesoporous neuronal electrode surface-modified with a thiol group according to the example of the present invention.

### DETAILED DESCRIPTION

Hereinafter, the present invention will be described in detail with reference to exemplary embodiments.

A mesoporous neuronal electrode according to the present invention includes a nanoparticle layer formed on a surface of the electrode and including at least one of a first metal nanoparaticle and a second metal nanoparticle selected from the group consisting of a nanotube, a hollow nanoparticle and a nanowire. The nanotube, the hollow nanoparticle and the nanowire are forms having a large surface area per unit area, which enable an increase in surface area to improve an electric current. The nanoparticle layer may have a thickness of 40 nm to 1 μm. When the thickness of the nanoparticle layer is smaller than 40 nm, surface modification is hardly effective. When the thickness of the nanoparticle layer is greater than 1 μm, performance of the neuronal electrode is not enhanced any longer in proportion to an increase in the thickness of the layer.

A first metal may include gold and a second metal may include white gold, or the first metal may include white gold and the second metal may include iridium. Gold, white gold and iridium metals are particles with a microcrystalline structure, in which metal nanoparticles formed by electro-co-depositing one or more metals have different sizes and thus may increase a surface area as compared with those formed by electro-co-depositing a single metal. An increase in surface area may reduce impedance of the electrode and increase capacitance of the electrode at the same time, contributing to not only enhancement of signal detection sensitivity but reduction in thermal noise.

The mesoporous neuronal electrode may have an impedance of 1×10⁷ or lower at 1 kHz.

When the impedance of the mesoporous neuronal electrode is higher than 1×10⁷ at 1 kHz, the mesoporous neuronal electrode may not properly work as a neuronal electrode since the electrode has a difficulty in measuring a signal.

The mesoporous neuronal electrode may have a capacitance of 1 mF/Cm² or higher. When the capacitance of the mesoporous neuronal electrode is lower than 1 mF/Cm², the mesoporous neuronal electrode may not properly work as a neuronal electrode.

The first metal nanoparticle, the second metal nanoparticle or both of the first metal nanoparticle and the second metal nanoparticle may be combined with a functional group. Preferably, the functional group is combined with the first metal nanoparticle. The functional group may include a metal, a polymer and hyalin which are chemically combined.

The functional group may be combined via self-assembly with the first metal nanoparticle, the second metal nanoparticle or both of the first metal nanoparticle and the second metal nanoparticle. Preferably, the functional group is combined via self-assembly with the first metal nanoparticle. Self-assembly is achieved by a covalent bond.

The functional group may be a bioaffinitive functional group. Since the functional group requires not causing side effects, such as adverse reactions, when used for a human body, the bioaffinitive functional group that works easily in neurons is preferably used.

The functional group may include a thiol group. Here, a compound including the thiol group may be at least one compound selected from the group consisting of 4-Mercaptobenzoic acid, 8-Mercaptooctanoic acid, 6-Mercaptohexanoic acid and 3-Mercaptopropionic acid.

A neural signal measuring apparatus according to the present invention includes the mesoporous neuronal electrode. Since the neuronal electrode may detect or stimulate neural signals inside or outside neurons without causing damage to the neurons, the neural signal measuring apparatus has excellence in biocompatibility and biostability.

A method of making a mesoporous neuronal electrode according to the present invention includes preparing a mixture solution including a first metal precursor solution, a second metal precursor solution and a surfactant, introducing a target electrode into the mixture solution and electro-co-depositing a first metal nanoparticle and a second metal nanoparticle on a surface of the target electrode, and washing the target electrode with the electro-co-deposited first metal nanoparticle and second metal nanoparticle to remove the surfactant and to form a carbon nanotube, a hollow nanoparticle or both of the carbon nanotube and the hollow nanoparticle including the first metal nanoparticle and the second nanoparticle.

In the electro-co-depositing, electrodeposition is generally known as a method of coating an electrode with metal nanoparticles with low costs. Electrodeposition employs a principle that a deposition target is transferred to a cathode or anode using a binder dissolved in a medium and the binder which becomes insoluble in the medium through a chemical reaction on the anode or cathode is deposited to coat the deposition target. Electrodeposition produces a coating layer with a uniform thickness and concentration, quickly forms a layer, easily adjusts the thickness and enables coating of an irregular object and thus is particularly utilized for technologies using nanoparticle-sized metallic materials.

A first metal precursor may include HAuCl₄ or KAuCl₄, and a second metal precursor may include H₂PtCl₆ or K₂PtCl₆. Preferably, the first metal precursor is HAuCl₄, and the second metal precursor is H₂PtCl₆.

The surfactant may be present in an amount of 0.1 to 30% by weight (wt %) in the mixture solution. When the amount of the surfactant is less than 0.1 wt %, a mesoporous structure is not properly formed. When the amount of the surfactant is greater than 30 wt %, a mesoporous structure excessively develops after the surfactant is removed, causing a problem for structural stability.

The surfactant may be at least one selected from the group consisting of P123, SDS, CTAB and Triton X. The surfactant is included in the mixture solution and removed after electro-co-deposition, thereby forming metal nanoparticles with a mesoporous structure.

Electro-co-deposition may be carried out by cyclic voltammetry or a constant voltage method. Generally, cyclic voltammetry is an analysis method used for studies of oxidation/reduction rates and mechanisms, particularly organic and metal-organic studies, which measures a change in amount of an electric current of a working electrode according to a voltage change while linearly changing voltage over time. Here, “cyclic” means changing voltage from an initial set voltage to a final set voltage over time and then changing voltage from the final set voltage back to the initial set voltage. Characteristics of ions put in samples are analyzed using the change in amount of the electric current according to the voltage change.

The method may further include introducing a function group to be combined with the first metal nanoparticle, the second metal nanopaticle or both of the first metal nanoparticle and the second metal nanoparticle after the washing. The functional group may be a bioaffinitive functional group and be combined via self-assembly.

The mesoporous neuronal electrode of the present invention is obtained by simultaneously electrodepositing gold and white gold nanoparticles having a mesoporous surface on the surface of the electrode using the surfactant and an electrochemical method, thereby reducing the impedance of the electrode and increasing the capacitance of the electrode. Also, the mesoporous neuronal electrode may be chemically combined with any material due to surface modification of gold nanoparticles with the bioaffinitive functional group, thus improving surface modification performance of the electrode. In addition, the electrode with improved surface modification performance allows neurons to stably grow to increase probability of detecting neural signals.

Hereinafter, the present invention is described in more detail with reference to the following example, but it should be noted that the present invention is not limited to the example.

### EXAMPLE

HAuCl₄ and H₂PtCl₆ were dissolved in sulfuric acid as a solvent to prepare a first metal solution and a second metal solution, respectively. The first metal solution and the second metal solution were mixed at a 1:9 ratio to prepare an electrolyte solution, after which 0.2 wt % of P123 as a surfactant was added to the electrolyte solution, thereby producing a mixture. An Ag/AgCl electrode in a KCI saturated solution as a counter electrode, a white gold-plate reference electrode and a working electrode were prepared, and the working electrode was dipped in the mixture. A constant voltage of −0.2 V as compared with the counter electrode was applied to the working electrode for 10 minutes using a scanning potentiostat (EG and G model 273A), thereby electro-co-depositing gold nanoparticles and white gold nanoparticles on the working electrode. The working electrode with the gold and white gold nanoparticles electro-co-deposited in the surfactant solution was taken out of a reactor and washed with distilled water to remove the surfactant.

The working electrode with the electro-co-deposited gold and white gold nanoparticles was combined with 4-Mercaptobenzoic acid having a thiol functional group using a self-assembled monolayers technique, thereby manufacturing a mesoporous neuronal electrode surface-modified with the thiol functional group.

### Compartive Example 1

A neuronal electrode was prepared by electrodepositing gold on a working electrode, instead of gold/white gold nanoparticles, and combining with the same thiol functional group as used in the example.

### Comparative Example 2

A neuronal electrode was prepared by electrodepositing nanoparticles on a working electrode using a precursor solution in the same manner as in the example, in which the precursor solution is a gold precursor solution, instead of the mixture solution, non-mesoporous gold nanoparaticles were electrodeposited on the working electrode without a process of mixing and removing a surfactant, and the same thiol functional group as used in the example is combined.

### Comparative Example 3

A neuronal electrode was prepared in the same manner as in the example except that non-mesoporous gold/white gold nanoparticles were electro-co-deposited on a working electrode using an electrolyte solution not including a surfactant.

### Experimental Example

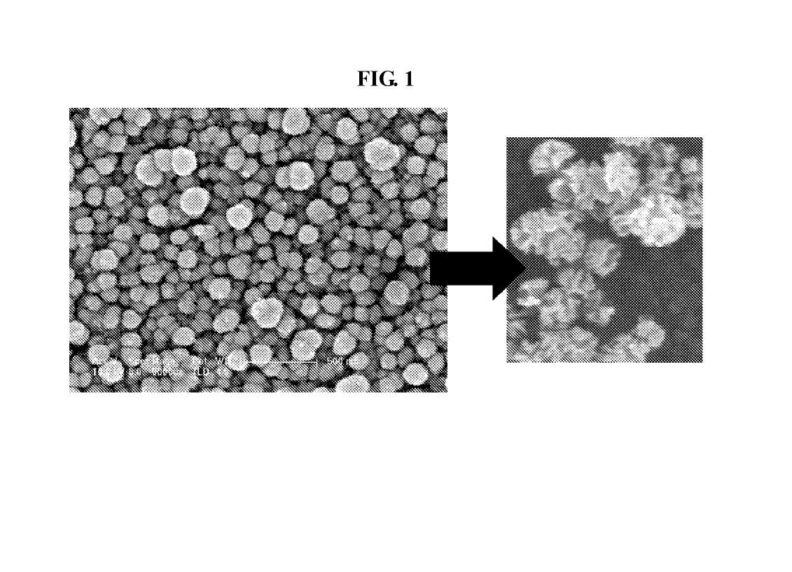

FIG. 1 is a scanning electron microscopy (SEM) of gold/white gold nanoparticles according to the example of the present invention. The gold and white gold nanoparticles have different particle sizes, which increase a surface area as compared with single metal nanoparticles. Further, the gold and white gold nanoparaticles are mesoporous-structure nanoparticles.

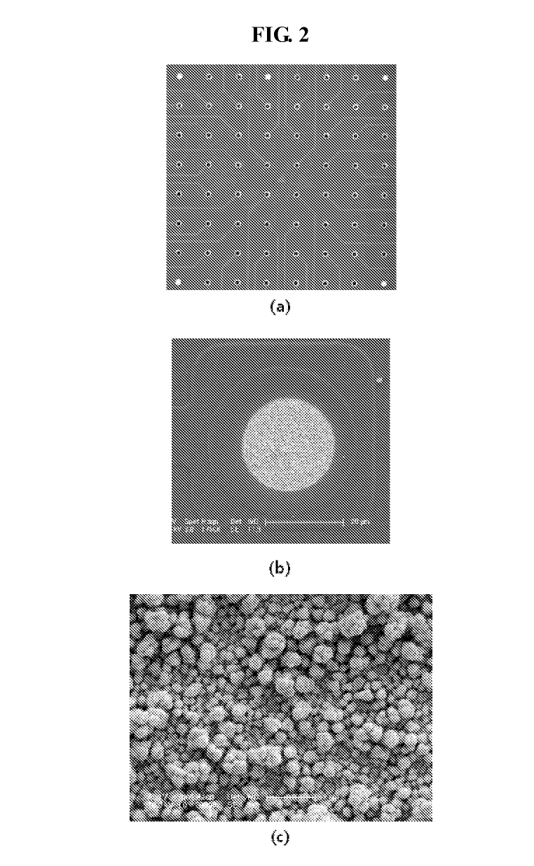

FIG. 2(a) is a light microscope image of the mesoporous neuronal electrode with electro-co-deposited gold/white gold nanoparticles according to the example of the present invention. The mesoporous neuronal electrode of FIG. 2(a) has a large surface area to reduce impedance of the electrode and to increase capacitance thereof, thus improving performance of the electrode.

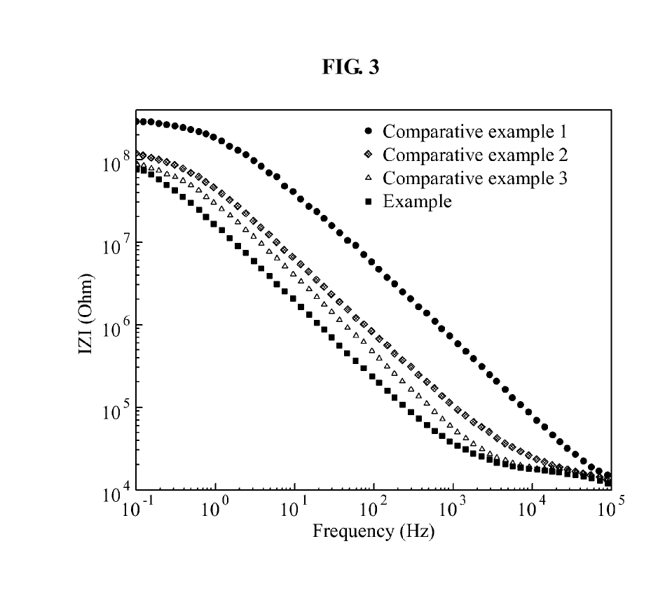

FIG. 3 is a graph illustrating impedances of the neuronal electrodes according to the example and the comparative examples 1 to 3. FIG. 3 shows that the mesoporous neuronal electrode including different kinds of nanoparticles according to the example exhibits a greater decrease in impedance than those according to the comparative examples 1 to 3, thus reducing electrical noise. Also, an increase in surface area due to the mesoporous structure formed by the surfactant contributes to the decrease in impedance.

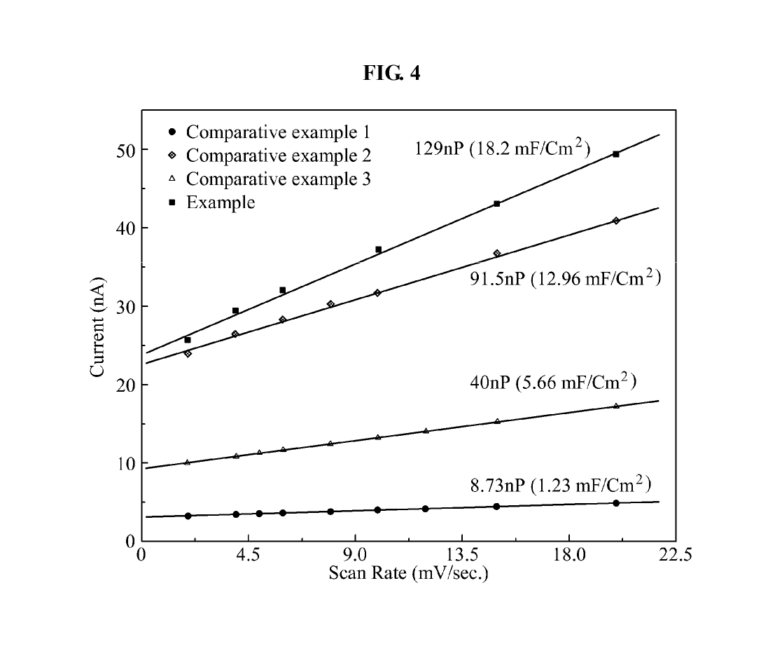

FIG. 4 is a graph illustrating capacitances of the neuronal electrodes according to the example and the comparative examples 1 to 3. FIG. 4 shows that the mesoporous neuronal electrode including different kinds of nanoparticles according to the example exhibits a greater capacitance than those according to the comparative examples 1 to 3. In particular, as compared with that according to the comparative example 3, the mesoporous neuronal electrode of the example has a high capacitance, which results from an increase in surface area due to the mesoporous structure.

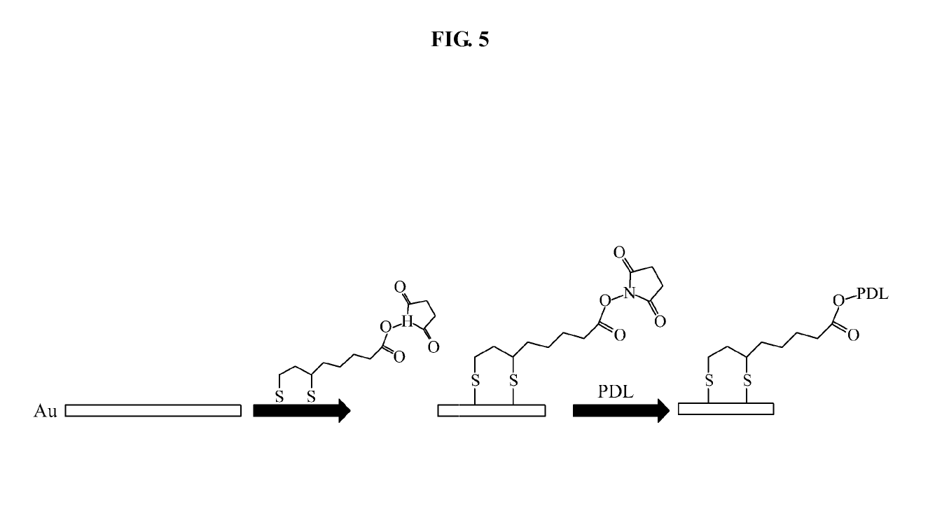

FIG. 5 schematically illustrates the mesoporous neuronal electrode surface-modified with a thiol group according to the example of the present invention. It is considered that the thiol group is combined with surface of mesoporous particles to enhance performance of the neuronal electrode.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A mesoporous neuronal electrode comprising:
a nanoparticle layer being formed on a surface of the electrode and comprising at least one of a first metal nanoparticle and a second metal nanoparticle selected from the group consisting of a nanotube, a hollow nanoparticle and a nanowire.

2. The mesoporous neuronal electrode of claim 1, wherein the nanoparticle layer has a thickness of 40 nm to 1 μm.

3. The mesoporous neuronal electrode of claim 1, wherein a first metal comprises gold and a second metal comprises white gold, or the first metal comprises white gold and the second metal comprises iridium.

4. The mesoporous neuronal electrode of claim 1, wherein the mesoporous neuronal electrode has an impedance of 1×10⁷ or lower at 1 kHz.

5. The mesoporous neuronal electrode of claim 1, wherein the mesoporous neuronal electrode has a capacitance of 1 mF/Cm² or higher.

6. The mesoporous neuronal electrode of claim 1, wherein the first metal nanoparticle, the second metal nanoparticle or both of the first metal nanoparticle and the second metal nanoparticle are combined with a functional group.

7. The mesoporous neuronal electrode of claim 6, wherein the functional group is combined via self-assembly with the first metal nanoparticle, the second metal nanoparticle or both of the first metal nanoparticle and the second metal nanoparticle.

8. The mesoporous neuronal electrode of claim 6, wherein the functional group is a bioaffinitive functional group.

9. The mesoporous neuronal electrode of claim 6, wherein the functional group comprises a thiol group.

10. A neural signal measuring apparatus comprising the mesoporous neuronal electrode of claim 1.

11. A method of making a mesoporous neuronal electrode, the method comprising:
preparing a mixture solution comprising a first metal precursor solution, a second metal precursor solution and a surfactant;
introducing a target electrode into the mixture solution and electro-co-depositing a first metal nanoparticle and a second metal nanoparticle on a surface of the target electrode; and
washing the target electrode with the electro-co-deposited first metal nanoparticle and second metal nanoparticle to remove the surfactant and to form a carbon nanotube, a hollow nanoparticle or both of the carbon nanotube and the hollow nanoparticle comprising the first metal nanoparticle and the second nanoparticle.

12. The method of claim 11, wherein a first metal precursor comprises HAuCl₄ or KAuCl₄, and a second metal precursor comprises H₂PtCl₆ or K₂PtCl₆.

13. The method of claim 11, wherein the surfactant is present in an amount of 0.1 to 30% by weight (wt %) in the mixture solution.

14. The method of claim 11, wherein the surfactant comprises at least one selected from the group consisting of P123, SDS, CTAB and Triton X.

15. The method of claim 11, wherein the electro-co-depositing is carried out by cyclic voltammetry or a constant voltage method.

16. The method of claim 11, further comprising introducing a function group to be combined with the first metal nanoparticle, the second metal nanopaticle or both of the first metal nanoparticle and the second metal nanoparticle after the washing.
